(19) United States
(12) Patent Application Publication
(10) Pub. No.: US 2006/0030809 A1
(43) Pub. Date: Feb. 9, 2006

(54) Apparatus and method for multiple organ assist

(76) Inventors: Amir Barzilay, Zur Hadassah (IL); Ascher Shmulewitz, Tel Aviv (IL)

Correspondence Address: PEARL COHEN ZEDEK, LLP, 10 ROCKEFELLER PLAZA, SUITE 1001, NEW YORK, NY, 10020, US

(21) Appl. No.: 11/178,603

(22) Filed: Jul. 12, 2005

Related U.S. Application Data
(60) Provisional application No. 60/586,710, filed on Jul. 12, 2004.

(51) Int. Cl. A61M 37/00

(52) U.S. Cl. 604/6.11

(57) ABSTRACT

An apparatus and method for multiple organ assist. The apparatus may include a cannula suitable for insertion into a blood system, the cannula having at least one inlet and at least one outlet situated at a predetermined distance from each other such that said inlet and outlet are located in proximity to a first organ and a second organ, respectively, when the cannula is inserted into said blood system; and a pumping mechanism able to pump blood though said cannula from said inlet to said outlet.

### CROSS REFERENCE TO PRIOR APPLICATION

This application claims priority and benefit from U.S. Provisional Patent Application No. 60/586,710, entitled “Apparatus and Method for Multiple Organ Assist”, filed on Jul. 12, 2004, which is hereby incorporated by reference in its entirety.

### FIELD OF THE INVENTION

The present invention relates to methods and devices useful in treating individuals suffering from temporary or permanent decrease in cardiac output. More specifically, the present invention relates to devices and methods for concurrent augmentation of cardiac output and the prevention of vital organ failure.

### BACKGROUND OF THE INVENTION

Individuals suffering from temporary or permanent reduction of their heart's cardiac output face severe health problems which frequently become fatal. Temporary reduction in cardiac output may be the result of an acute cardiac event or trauma, from which the patient can sometimes recover within a relatively short period of time. Permanent reduction of cardiac output is typically the result of CHF (Congestive Heart Failure) or DHF (Diastolic Heart Failure). In the USA alone, there are in excess of 6 Million CHF and DHF patients, and 400,000 new CHF/DHF patients are added each year.

A decrease in a person's cardiac output may negatively affect the provision of quality oxygenated blood to many vital organs, for example, the kidneys. As a result, the organs' prime functions may be materially impaired. In the case of the kidneys, the removal of blood toxins and excess body fluids removal through urination may be impaired, resulting in excess fluids being aggregated in the body, causing further overload on the already failing heart and to further decreases in renal function. As many as 30% of the mortality episodes in intensive care units are the outcome of renal failure resulting from deteriorating CHF/DHF patients.

There are a variety of known devices and methods for the treatment of patients suffering from either one or a combination of decreased cardiac output, aggregation of excess body fluids and renal insufficiency or failure

One currently used heart assist device is an implantable blood pump, which is inserted into the patient's chest via surgery. Such an implantable heart assist device receives its power from batteries outside the patient's body. The surgery required for such device is quite complex, and the device is typically very expensive. Such a device is often utilized as a bridge to heart-transplant for end-stage CHF/DHF patients who may not otherwise survive.

Another type of heart assist device is an extra corporeal blood pump, which provides cardiac output augmentation functions and is typically used in bypass surgery and as a bridge-to-recovery solution for patients recovering from cardiac events, or for CHF/DHF patients suffering from deterioration of their chronic condition. Such extra corporeal blood pumps generally require the insertion of two catheters into the patient's body (one for withdrawing blood and one for returning blood).

Currently used kidney assist devices include Hemodialysis, Hemofiltration and Ultrafiltration devices. Such devices remove toxins and excess fluids from the circulating blood. The principle behind these devices is generally that the device augments (and frequently replaces) failing renal function by removing excess fluids, thereby reducing the overload on the failing heart, to enable revitalized cardiac output that aims to improve renal perfusion and renal function. It should be noted that Hemofiltration cannot typically be implemented on instable patients.

Additional kidney assist devices relate to the localized delivery of anti-dilatation drugs into the renal arteries. One of the immediate outcomes of reduced cardiac output is the contraction of the renal arteries. As a result, even the reduced cardiac output generated by the failing heart cannot reach the kidneys. The use of special catheters specifically designed for localized delivery of anti-dilatation drugs into the renal arteries is aimed at improving this situation. It should be noted that the renal drug delivery catheter requires a minimally invasive procedure during which there is a significant probability that the renal artery or arteries may be punctured.

A further type of kidney assist devices relate to provision of systemic drugs, such as anti-dilating drugs, diuretic drugs and other drugs. In order to be effective, the dosages required are quite high, often resulting in adverse reactions.

Because none of the above solutions is optimal, even without taking into account high cost, invasive insertion procedures and risks associated with the current state-of-the-art procedures, caregivers typically use several of these devices and drugs concurrently in order to improve a patient's condition and save the patient from a fatal occurrence. A treatment protocol of a deteriorated CH/DHF patient often includes an intra-aortic balloon pump, periodic Hemofiltration for excess fluids removal, and the provision of systemic diuretics and anti-dilatation drugs. It is evident that simply maintaining the patient balanced under this complex treatment regime may be a complicated and cost-ineffective task

Thus, new treatments are desired to more simply and cost effectively perform the variety of functions involving failing heart assist, cardiac output augmentation, excess fluids removal, renal arteries dilatation through localized drug delivery and improved renal perfusion, to timely balance and improve the patient's health condition and to avoid the patient's deterioration into an irreversible fatal condition.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a multiple organ assist apparatus according to an exemplary embodiment of the present invention;

FIG. 2A is a schematic illustration of a multiple organ assist apparatus inserted into a human body, according to exemplary embodiments of the present invention;

FIG. 2B is a schematic illustration of a multiple organ assist apparatus inserted into a human body, according to further exemplary embodiments of the present invention;

FIG. 3 is a schematic illustration of a cannula and fluid reservoir, according to some embodiments of the present invention;

FIG. 4 is a schematic illustration of a cannula and fluid reservoir, according to other embodiments of the present invention;

FIG. 5A is a schematic illustration of a fluid reservoir associated with various sub-systems of an organ assist apparatus according to some embodiments of the present invention;

FIG. 5B is a schematic illustration of an organ assist apparatus including an Oxygenation sub-system, according to some embodiments of the present invention, and

FIG. 6 is a flowchart of a method of providing concurrent multiple organ assist, according to exemplary embodiments of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The phrases “heart and kidney assist apparatus”, “heart and vital organ assist apparatus”, “blood pumping apparatus”, “blood assist apparatus” or “multiple organ assist apparatus”, as used herein, may encompass cardiac assistant devices, systems, apparatuses and methods, and/or any other suitable devices, systems, apparatuses and methods for assisting bodily organs by impacting at least partially on heart output. The term “cannula” as used herein may encompass catheters, tubes, or other suitable mechanisms to be inserted into a bodily cavity to drain fluid or administer substances, etc. The terms “heart”, “kidney”, “aorta” or usage of other particular organs or lumen etc. as used herein, may encompass the organs or lumen mentioned, or a location, vessels etc. substantially in proximity to the particular organ or lumen etc.

According to some embodiments of the present invention, an apparatus is provided that is capable of concurrently providing one or more of cardiac failing heart assistance, cardiac output augmentation, organ perfusion, removal of excess bodily fluids, localized delivery of drugs, extra corporeal blood oxygenation, and/or other suitable bodily assist functions for two or more organs.

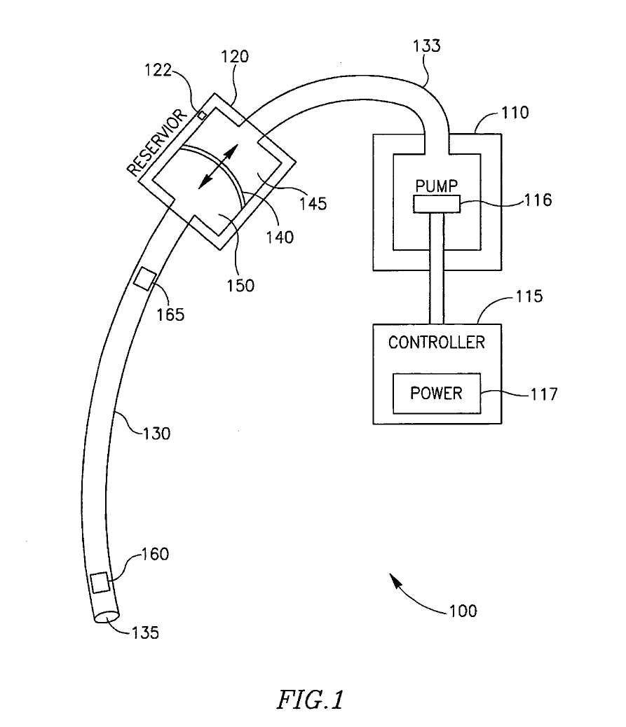

Reference is now made to FIG. 1, which illustrates a multiple organ assist apparatus 100 according to an exemplary embodiment of the invention. Apparatus 100 may include an extra corporeal pumping mechanism, which may include, for example, a blood pump 110 and a fluid reservoir 120. Apparatus 100 may include a cannula 130, which may include at least one inlet, which may include, for example, an inlet valve 160, and at least one outlet, which may include, for example, an outlet valve 165. Inlet valve 160 and outlet valve 165 may be situated at a predetermined distance from each other such that the inlet and outlet valves are located in proximity to a first organ and a second organ, respectively, when the cannula is inserted into the blood system. The fluid reservoir 120 may be located between a proximal end 155 of cannula 130 and blood pump 110. Fluid reservoir 120 may be connected to blood pump 110 by a tube, catheter, or cannula etc. 133. Reservoir 120 may include at least one outlet 122, for at least partially removing contents from reservoir 120.

Blood pump 110, which may be, for example, a pulsatile hydraulic pump, may enable increasing and decreasing of fluid volume in a proximal compartment 145 of reservoir 120, in coordination and synchronization with the patient's heart, thereby to produce, for example, a pulsatile pumping action of blood to/firm a blood source. For example, blood may be pumped from a blood source (e.g., the aorta) to reservoir 120, through cannula 130. Blood may additionally be pumped from reservoir 120 to a blood source (e.g., the aorta), through cannula 130. Pump 110 may thus assist in pumping blood through the body of a patient at, for example, sub-cardiac volumetric rates, or other suitable rates, as described below. Pump 110 may be a pulsatile pump, rotary pump, peristaltic roller blood pump, centrifugal blood pump, or any other suitable type of pump.

Reservoir 120 may include at least a proximal compartment 145 and a distal compartment 150, separated by at least one flexible diaphragm 140. Diaphragm 140 may be constructed, for example, from semi-permeable or non-permeable material. The proximal compartment 145 of the fluid reservoir may be connected to pump 110 and may enclose fluid within it, for example, a substantially non-compressible fluid. The distal compartment 150 of fluid reservoir 120 may be connected to proximal end 155 of cannula 130. The distal end of cannula 135 may be positioned at a selected location in the body, for example, a blood source.

Cannula 130 may enable the inlet and/or outlet valves to be opened and/or shut in response to, for example, pressure changes in cannula 130 resulting from operation of pump 110. Diaphragm 140 may deform to alter the respective volumes of compartments 150 and 145, so as to substantially control the flow of fluid or blood respectively in the two compartments. Diaphragm 140 may also prevent mingling of the fluids in the two compartments. Proximal compartment 145 may contain a substantially incompressible liquid, e.g., water, saline solution, or any other suitable fluid. Since compartment 145 may be coupled to pump 140, its volume may increase or decrease in response to “up” or “down” motion, respectively, of a pumping mechanism, for example, a piston 116 in pump 110, thereby to correspondingly increase or decrease the fluid pressure in compartment 145. For example, by decreasing pressure in compartment 145, blood may be pumped into inlet valve 160, and by increasing pressure in compartment 145, blood may be pumped out of outlet valve 165.

Pump 110 may be controlled by an internal computer or controller 115, which may regulate the rate and stroke volume of piston 116. Controller 115 may receive physiological signal inputs, such as ECG and/or blood pressure signals, and may use these signals in controlling pump 110. A power source 117 may be provided for controller 115, pump 110, and/or other elements of apparatus 100.

Controller 115 may enable adjusting a delay of piston 116 stroke relative to the systolic and/or diastolic stroke of the heart. This delay may be adjusted, for example, so that cannula 130 pumps blood out synchronously with the heart's systole; counter synchronously, during diastole; or at any suitable phase there between. In other embodiments the rate of piston 116 may be set to be independent of the heart rate, for example in order to maintain steady perfusion during arrhythmia or fibrillation. The controller may enable customization and/or programming of the operation of multiple organ assist apparatus 100, to provide functionality suitable to each particular patient, each type of patient, and/or particular medical scenarios.

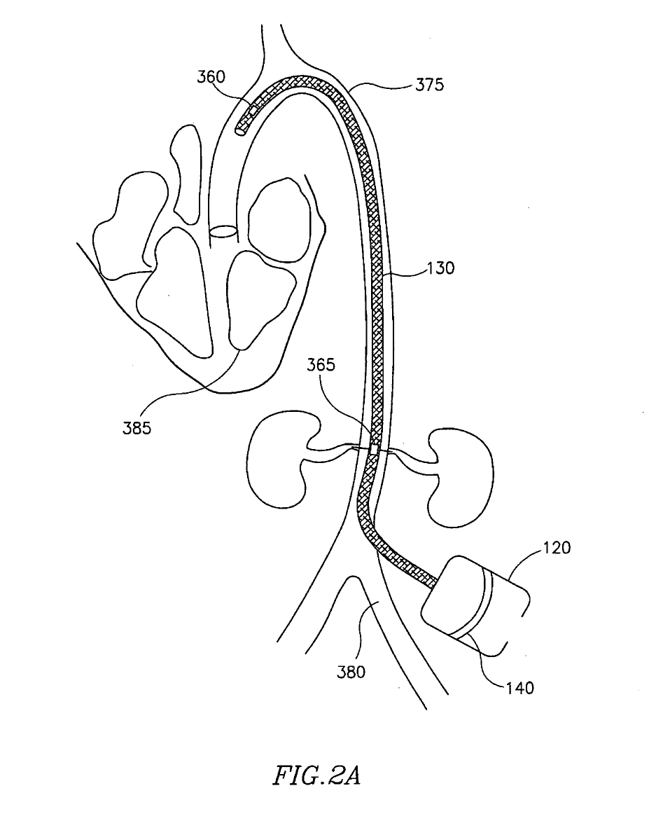

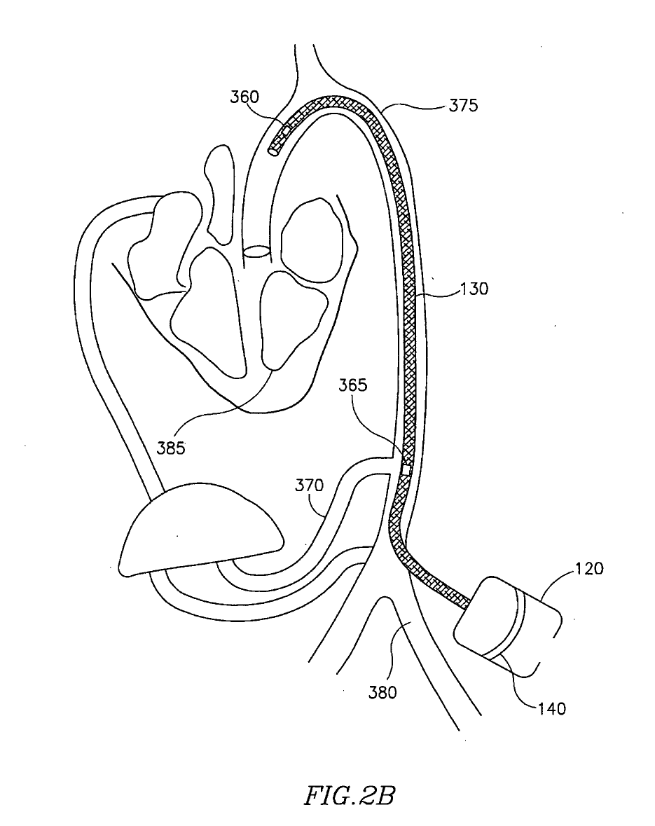

Reference is now made to FIG. 2A, which schematically illustrates cannula 130 inserted into a human body with distal end 135 brought to a desired position in proximity to the human heart, for example, in the aorta, according to an exemplary embodiment of the present invention. Inlet valve 360 is indicated in the upper aorta, and at least one outlet valve 365 is indicated, for example, in proximity to a renal artery or both renal arteries. This exemplary configuration may be suitable for the treatment of a failing heart, cardiac output augmentation, organ perfusion, the removal of excess bodily fluids, localized delivery of drugs, extra corporeal blood oxygenation and/or other conditions, by pumping blood from a blood source to at least one selected location, for example, from the heart to the kidneys.

Reference is now made to FIG. 2B, which schematically illustrates cannula 130 inserted into a human body with distal end 135 brought to a desired position in proximity to the human heart, for example, in the aorta, according to an exemplary embodiment of the present invention. Inlet valve 360 is indicated in the upper aorta, and at least one outlet valve 365 is indicated, for example, in proximity to the Hepatic artery 370. In other, embodiments outlet valve 365 of cannula 130 may be placed in proximity to other suitable organs, lumens, vessels etc.

In some embodiments cannula 130 may be formed of flexible material. To administer treatment, cannula 130 may be entered into a blood vessel of a patient at distal end 135, and proximal end 155 may be connected to the distal end 150 of fluid reservoir 120. One or more inlet valves 160 and one or more output valves 165 may be provided, and each of the input and/or output valves may be designed in a different manner

In some embodiments of the present invention, at least one inlet valve 160 is a unidirectional valve situated in or in proximity to distal end 135 of cannula 130, which may allow flow into the cannula and prevent flow out of the cannula. Through this at least one inlet valve 160, which may be open when the pressure within cannula 130 is decreased, blood may flow from the blood source into cannula 130. When the pressure in cannula 130 is increased, the at least one inlet valve 160 may be closed, and the at least one outlet valve 165 may be opened, thereby allowing blood to return through outlet valve 165 to a selected location. Inlet valve 160 and outlet valve 165 may be any suitable types of one-way valve, for example mechanical flap valves, leaflet valves etc.

In some exemplary embodiments of the present invention, the at least one outlet valve 165 may be a unidirectional valve situated between the distal end 135 and proximal end 155 of cannula 130, which may allow blood flow out of the cannula to a target location, and prevent blood flow into the cannula through outlet valve 165. When the pressure in cannula 130 is decreased, the at least one outlet valve 165 may be closed, so that blood from the blood vessel in which cannula 130 is inserted cannot flow out of cannula 130. When the pressure in cannula 130 is increased, the at least one outlet valve 165 may be opened, and blood from cannula 130 may flow out of cannula 130 into the target location, for example, a blood vessel or lumen proximal to where outlet valve 165 is positioned.

In some embodiments, for example, where kidney assist functions are to be implemented, outlet valve 165 may be situated on cannula 130, for example, between 30-60 cm from inlet valve 160, to enable blood to be withdrawn from the region of the aorta, and returned to the region of the renal arteries. In other embodiments, for example, where liver assist functions are to be implemented, outlet valve 165 may be situated on cannula 130, fox example, between 40-70 cm from inlet valve 160, to enable blood to be withdrawn from the region of the aorta, and returned to the region of the liver. Other organ assist functions may be implemented by suitably locating the inlet and outlet valves, such that the two valves may be located in different organs, or in proximity to different organs. For example, when cannula 130 is positioned in the body, with its distal end 135 in the aorta, output valve 165 may be situated outside the heart region, for example, in proximity to the renal artery, or any other suitable location or locations, depending on specific applications. According to some embodiments of the present invention, a multiple outlet cannula may be used, to transfer blood or other fluids between more than two organs or vessels.

In some embodiments of the present invention, distal end 135 of cannula 130 and the location of outlet valves 165 in cannula 130 may incorporate mm-kings and/or materials which enable the proper positioning of these elements within the target blood vessel using a variety of imaging technologies, for example, Ultrasound, CT and/or MRI etc., as are known in the art.

In some embodiments of the present invention, cannula 130 may be adapted to concurrently provide cardiac output augmentation and organ perfusion. The length of cannula 130 may be designed such that, after insertion through a femoral entry, the distal end 135 of cannula 130 may be positioned in the left ventricle or the aortic arch, or in the upper segment of the aorta in proximity to the aortic arch. Other designs may be used, for other functions. The at least one inlet valve 160 may be situated in or in proximity to the distal end 135 of cannula 130, so that blood may be pumped into cannula 130 from, for example, the left ventricle and/or the aorta. The relative positions of inlet valve 160 and outlet valve 165 may be such, that when distal end 135 of cannula 130 assumes its desired position, the at least one outlet valve 165 may be positioned near a desired perfusion site, for example near the junction between the aorta and the renal arteries. When using such an embodiment, blood may be withdrawn from the left ventricle and/or aorta, and by that assisting in reducing the overload on the heart. When the pressure in cannula 130 increases, blood may flow at a relatively high pressure directly into the renal arteries, via output valve 165. Such an embodiment may thereby significantly improve kidney perfusion, and also assist in increasing the blood volume flowing in the descending aorta, and by that, assist in augmenting cardiac output.

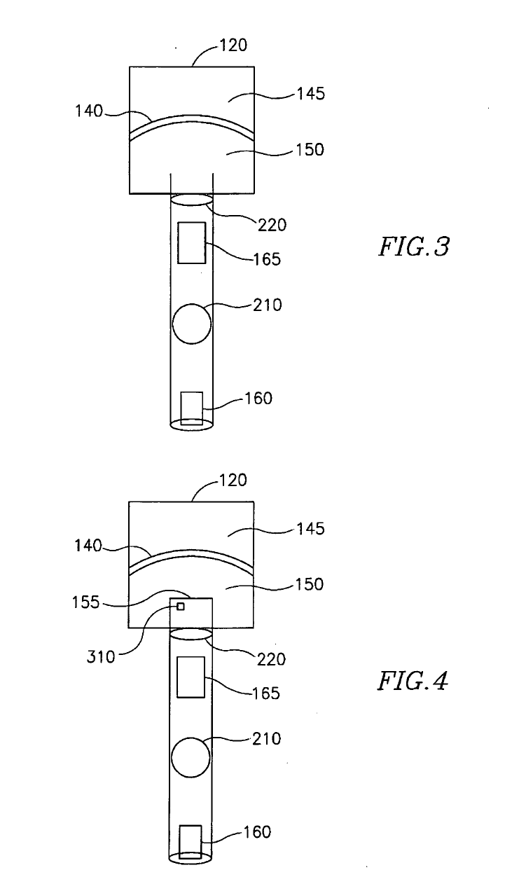

In some embodiments of the present invention, as can be seen with reference to FIG. 3, cannula 130 may farther incorporate at least one inflatable balloon 210, which may be located between outlet valve 165 and distal end 135 of cannula 130. In some embodiments, at least one additional inflatable balloon 220 may be located between outlet valve 165 and proximal end 155 of cannula 130. The purpose of the at least one balloon, and in some embodiments at least two balloons may be to temporarily isolate a part of the blood vessel in which cannula 130 is inserted. The inflation and deflation of the at least one balloon can be fully coordinated with the patient's heart rate (e.g. deflated during systole and inflated during diastole), or inflated and deflated in partial coordination with the patient's heart late. For example, a balloon may be deflated with each systole, but inflated only with every second diastole, or inflated and deflated independently of the patient's heartbeat.

In some embodiments of the present invention, cannula 130 may be insertable into the aorta via a femoral access, and may be designed to concurrently provide failing heart assist, cardiac output augmentation, and/or renal perfusion. Cannula 130 may incorporate two or more balloons, one balloon located between the outlet valves and cannula 130's distal end, and one balloon located between the outlet valves and cannula 130's proximal end. During systole, the pressure within cannula 130 may be decreased, and by deflating both balloons when blood flows into cannula 130 from the left ventricle, aortic arch or the upper aorta (when the outlet valves are closed), the function of failing heart assist may be performed. When the pressure in cannula 130 increases, both balloons may be inflated, segmenting the aorta above and/or below a selected aortic junction (e.g., g., the junction where the aorta meets the renal arteries), so that blood flows at relatively high pressure directly into one or more selected arteries (e.g., the renal arteries) to help facilitate improved organ perfusion. As the balloons are inflated during diastole, and as the blood is returned from cannula 130 back to the descending aorta, cannula 130 may concurrently provide assistance to the failing heart, augmentation of cardiac output, and improved organ perfusion.

Reference is now made to FIG. 4, which illustrates a cannula 130 that may incorporate at least one inlet valve 160, at least two outlet valves, e.g., outlet valve(s) 165 and an additional outlet valve(s) 310, and at least two balloons 210, 220, according to some embodiments of the present invention. In this configuration of cannula 130, the at least one additional outlet valve, for example two additional outlet valves 310, may be located between balloon 220 and proximal end 155 of cannula 130. The additional outlet valves 310 may enable desired distribution of the returned blood between the organ perfusion operation and the descending aorta perfusion operation. The nature and size of additional outlet valves 310 may be different from the perfusion outlet valves 165, so that different blood return profiles may be obtained.

In some embodiments of the present invention, the inner part of cannula 130 and/or the inlet valves 160 and outlet valves 165, 310, may be coated with one or more chemical substances, or, a combination thereof, facilitating reduction of hemolysis and other blood damage phenomena. Such chemical compounds may include biocompatible materials similar in principle to the inner coating of human blood vessels, anti-coagulation compounds, or other suitable substances. The inner coating of cannula 130 may cause the elution of one or more of the incorporated chemical compounds, in such a manner that the protection provided by the compounds may be sustained for at least as long as cannula 130 is in the patient's blood vessel.

In some embodiments of the present invention, fluid reservoir 120 located between cannula 130 and pulsatile blood pump 110, may enable the pressure in compartment 145 of fluid reservoir 120, to be changed. For example, a reduction in pressure in compartment 145 may cause blood to flow from the blood vessel through cannula 130 into compartment 150 of fluid reservoir 120. An increase in pressure in compartment 145 may cause blood to flow from compartment 150 to a selected destination through cannula 130, for example, via outlet 165.

In some embodiments of the present invention, fluid reservoir 120 and/or additional sub-systems integrated within it, may be capable of performing at least one or a combination of removal of excess fluids from the blood fluid withdrawn through cannula 130 into distal compartment 150 proximal compartment 145 of reservoir 120; adding one or more drugs into the blood fluid in distal compartment 150 for local delivery through the outlet valves of cannula 130; and/or for extra corporeal oxygenation of the patient's blood in distal compartment 150 of fluid reservoir 120.

In some embodiments of the present invention, diaphragm 140 separating the two compartments of fluid reservoir 120 may be constructed from semi-permeable materials designed to enable blood filtration (e.g. the removal of water or other substances but not important blood ingredients). The fluid in proximal compartment 145 below the diaphragm may be a dialysate fluid similar in nature to dialysate fluids used in standard ultrafiltration of the blood of patients suffering from ARI (Acute Renal Insufficiency), ARF (Acute Renal Failure) and ESRD (End Stage Renal Disease), or other suitable fluids. Under this design, the diaphragm may be capable of concurrently delivering the inflow and outflow pressures necessary to cause blood to flow into and out of proximal compartment 145, and to enable excess fluids from the blood to pass through the diaphragm (e.g. a semi-permeable ultrafiltration membrane), thereby helping to reduce the patient's blood volume without or by minimally affecting the blood vital compounds. In some embodiments chemicals, fluids, compounds or agents etc. may be used to aid filtration. In some embodiments, reverse osmosis may be used to extract excess fluids from the blood in compartment 150.

In the case where, during the operation of the multiple organ assist apparatus, the volume of the fluid in the proximal compartment 145 (e.g., containing the dialysate) is increased (e.g., in parallel to the patient's blood volume decreasing), there may be a need to periodically remove the excess fluid, so that, for example, the characteristics of the operation of fluid reservoir 120 shall not be negatively affected. Such removal of excess fluids from proximal compartment 145 of fluid reservoir 120 may be done using a liquid removal sub-system, for example, including a semi-permeable diaphragm 140. The liquid removal sub-system may enable liquid removal manually through a manual valve, automatically by a unidirectional valve located in proximal compartment 145 (e.g., which may be opened when the pressure in proximal compartment 145 exceeds a predefined value), or through a software and electronics controlled unidirectional valve, where such software and electronics are connected to one or more sensors measuring different parameters of at least one of pump 110, fluid reservoir 120 and cannula 130. According to some embodiments of the present invention, reservoir 120 and/or compartment 145 of reservoir may be a disposable element such that when a selected volume of excess fluid has been extracted from the blood, the reservoir or compartment may be replaced to enable more excess fluid to be extracted. In other embodiments at least one outlet 122 may be provided in reservoir 120 to enable extraction of excess fluid. In some embodiments a sensor may be provided to detect and optionally provide an alert when a selected quantity of excess fluid has been extracted from the blood. The sensor may enable excess fluid to be automatically released from outlet 122 or from other suitable outlet mechanisms.

Each patient may require a different amount of excess fluids to be removed from his/her blood circulation within a selected period of time (e.g., which may be measured in groups of 1 hours, 2 hours or 12 hours etc.). As a result, apparatus 100 according to embodiments of the present invention may be capable of removing, for example, up to 8,000 cc of excess fluids within each 8 hours of operation, but is capable of removing alternative (higher or lower) volumes per the requirements of the medical staff. The controlled variability in the rate of excess fluids removal is a result of at least one or a combination of the different permeability characteristics of the diaphragm, different compositions of dialysate fluid, the characteristics of excess fluid removal from the proximal compartment 145, the renewal of quality dialysate in the proximal compartment 145, and other factors.

In some embodiments, as the filtration operation continues, the quality of the dialysate may be reduced. A visual indication to the medical staff may be provided, for example, when the quality drops below a desired threshold by change of color of the dialysate fluid. Additionally or alternatively, a control system connected to proximal compartment 145 may sense the level of quality of the dialysate, as well as sense the quantity of fluids in this compartment, and automatically control the release of fluids through the unidirectional outlet valve, while inserting new quality dialysate back to the proximal compartment 145.

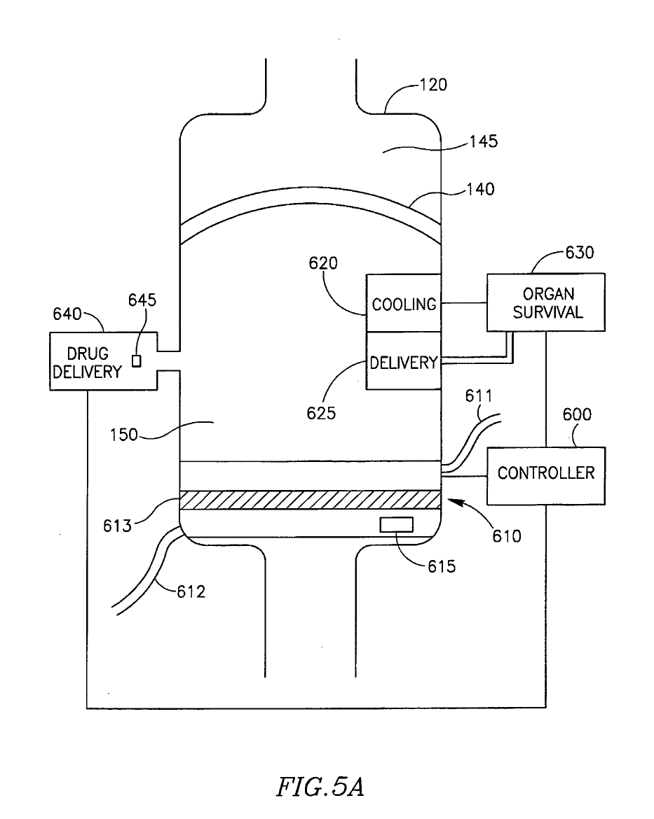

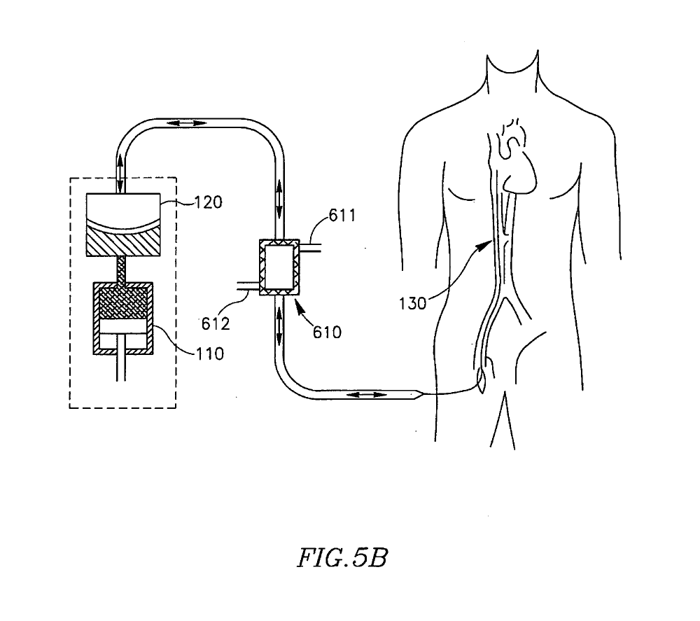

Reference is now made to FIG. 5A, which illustrates a fluid reservoir 120 associated with various sub-systems of an organ assist apparatus 100, according to some embodiments of the present invention. Fluid reservoir 120 may be associated with a blood oxygenation sub-system 610 for extra corporeal oxygenation of the patient's blood. The integration of such a sub-system may include integration of a gas exchange unit within the part of the reservoir containing the blood, integration of a bypass from the catheter into the reservoir compartment containing the blood, and/or integrating the gas exchange component within the catheter. Sub-system 610 may include a sensing and control unit 615, which may maintain pre-defined blood oxygen levels, and may prevent the entry of bubbles (e.g. generated as a part of the oxygenation process) into the blood stream of the patient

The blood oxygenation sub-system 610 may be capable of oxygenating both arterial blood and blood taken out of a vein, as is known in the art. With sub-system 610 in use, patients entering a low blood saturation episode, which cannot be treated with inhaled oxygen, may be treated without the need to employ heart lung machines or ECMO (Extra Corporeal Membrane Oxygenation) machines. A controller 600 may be provided to enable, for example, detecting of bubbles in the blood, monitoring and controlling the blood flow rate through blood oxygenation sub-system 610, and/or other suitable functions.

According to some embodiments of the present invention, blood oxygenation sub-system 610 may be associated with reservoir 120, for example, at the entry point of reservoir 120, to enable blood entering reservoir 120 to be oxygenated. Oxygenation sub-system 610 may include a gas inlet 611, for example, to enable entry of Oxygen into Oxygenation sub-system 610. Oxygenation sub-system 610 may include a gas outlet 612, for example, to enable exit of gasses (e.g., CON) from Oxygenation sub-system 610. Oxygenation sub-system 610 may include a membrane 613 to enable extraction of CO₂ from the blood in Oxygenation sub-system 610.

In other embodiments, as can be seen with reference to FIG. 5B, oxygenation sub-system 610 may be configured at a variety of locations on cannula 130. For example, sub-system 610 may be positioned within or associated with cannula 130, to be located externally or internally to the patient's body when catheter 130 is entered into the patient's body. For example, sub-system 610 may be positioned extraneously to the body, or may be positioned at a location within cannula 130, proximal to the blood source, for example, close to the aorta. For example, sub-system 610 may connect to cannula 130 at a location proximal to the blood target, for example, close to the renal artery or other suitable locations. Gas inlet 611 and gas escape outlet 612 may be located, for example, external to the entry point of cannula 130 into the body. In other embodiments sub-system 610 may connect to cannula 130 at a location close to the proximal end of cannula 130.

In some embodiments of the present invention, fluid reservoir 120 may be associated with an organ survival sub-system 630 aimed at facilitating extended organ(s) survival until harvesting such organ(s) for transplantation. The organ-survival sub-system 630 may enable lengthening, for example, for up to 24-48 hours, the viability of vital organs where the potential for patient fatality is high. The organ-survival sub-system may be associated with a temperature reduction component 620, for example liquid nitrogen filled tubes, capable of reducing the temperature of the patient's blood in reservoir 120, and a delivery component 625 capable of selectively delivering to one or more target vital organs cryo-precipitate fluids aimed at lengthening the period in which the target organ can remain viable for, harvesting, as are known in the art. The organ-survival sub-system 630 may include a control unit, or may be connected to controller 600, to facilitate accurate control of blood reduction temperatures, maintenance, and/or the delivery of the cryo-precipitate fluid(s) to the target organs that need to be harvested later.

In some embodiments of the present invention, fluid reservoir 120 may be associated with a sub-system for the localized delivery of drugs 640. The number of drugs which may be delivered by this drug delivery sub-system 640 may be unlimited. The drug delivery sub-system 640 may utilize the outlet valves 165 of cannula for delivery of drugs in a localized manner, thereby reducing the delivered drug concentrations (in comparison with systemic delivery). An example for such drug is a dilatation drug delivered to the kidney arteries in order to facilitate greater blood flow into the kidneys. Other suitable drugs may be administered. The output of the drug delivery sub-system 640 may be utilized in a variety of methods. For example, drug delivery sub-system 640 may be connected into distal compartment 150 of reservoir 120, i.e., where blood is contained, or into the catheter, or into any other suitable locations. The drug delivery sub-system 640 may enable unidirectional delivery of appropriate drugs. For example, when the pressure in the distal compartment 150 of reservoir 120 is decreased, a unidirectional valve 645 in the chug delivery sub-system 640 may be opened, enabling the drug(s) to flow and integrate in the blood in distal compartment 150. being pushed through the outlet valves of cannula 130. When the pressure in the distal compartment 150 of reservoir 120 is increased, the drug(s) may be delivered with the blood that is pushed back through the cannula 130 via, for example, outlet valve 165. The drugs delivery sub-system may further include an automated control sub-system which may be capable to concurrently control the delivery of multiple drugs, each chug with a different delivery protocol. Controller 600 may provide control signals or other means to control the administering of drugs by subsystem 640.

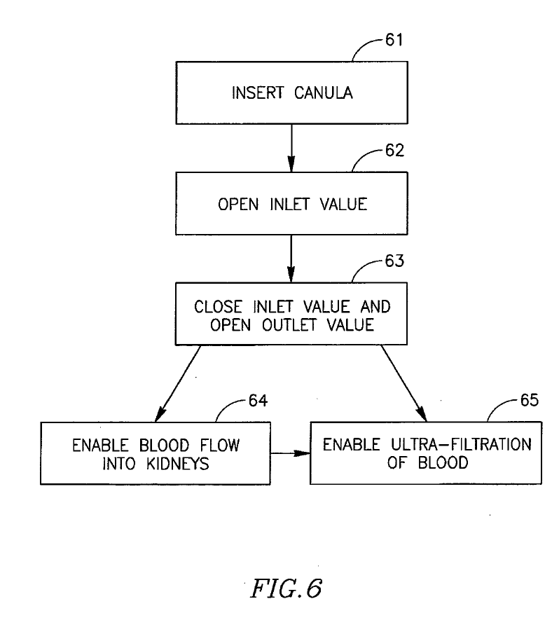

FIG. 6 schematically illustrates a series of operations or processes that may be implemented according to some embodiments of the present invention. As can be seen in FIG. 6, at block 61 cannula 130 may be inserted percutaneously, through an incision into a peripheral artery, for example the femoral artery 380, and passed upstream through aorta 375 into the aortic arch or upper aorta. The method of insertion may be substantially similar to methods for insertion of other types of cardiac cannulae known in the art. The length of cannula 130 may be approximately 60 cm, or any other suitable size, such that when distal end 135 is positioned in the aortic arch or upper aorta, proximal end 155 remains outside the body, adjacent to the incision. Alternatively, the cannula may be inserted surgically through a suitable incision elsewhere in the arterial system, and in such cases may be shorter than 60 cm, depending on the distance from the incision to the aortic arch or upper aorta. Other lengths may be used.

At block 62, once cannula 130 is in place, the inlet valve 160 may be opened thereby causing blood to flow from the aortic arch or upper aorta into cannula 130. Outlet valve 165 may be kept closed while the blood fills cannula 130. Blood flow into cannula 130 may be aided by pump 116 being withdrawn from blood pump 110.

At block 63 inlet valve 160 may be closed and outlet valve 165 may be opened. Blood flow out of cannula 130 may be aided by pump 116 being inserted into blood pump 110.

At block 64 the blood may flow out of cannula 130 and into a selected lumen. For example, in the case where outlet valve 165 is located substantially adjacent to the renal artery, the outlet valve 165 may enable blood to flow out of cannula 130 and into the kidneys. Any combination of the above steps may be implemented. Further, other steps or series of steps may be used.

At block 65, excess fluid in the blood inside multiple organ assist apparatus 100 may be extracted using filtration, for example, using a semi-permeable diaphragm (e.g., g., reverse osmosis) and/or ultrafiltration of substances or agents.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. An apparatus comprising:
a cannula suitable for insertion into a blood system, the cannula having at least one inlet and at least one outlet situated at a predetermined distance from each other such that said inlet and outlet are located in proximity to a first organ and a second organ, respectively, when the cannula is inserted into said blood system; and
a pumping mechanism able to pump blood through said cannula from said inlet to said outlet.

2. The apparatus of claim 1, wherein said pumping mechanism includes an extra corporeal blood pump and a fluid reservoir, wherein said fluid reservoir comprises a proximal compartment associated with said pump and a distal compartment associated with a proximal end of said cannula.

3. The apparatus of claim 1, wherein the distance between said inlet and said outlet is such that, when said cannula is inserted into a human body, said inlet valve is situated in the vicinity of the heart, and said output valve is situated in the vicinity of the kidneys.

4. The apparatus of claim 1, further comprising a controller to control the operation of said pumping mechanism.

5. The apparatus of claim 1, further comprising at least one balloon situated in said cannula.

6. The apparatus of claim 5, wherein said at least one balloon is located between said inlet and said outlet.

7. The apparatus of claim 5, comprising a plurality of balloons located on both sides of said outlet.

8. The apparatus of claim 1, wherein said cannula comprises two or more outlet valves near a proximal end of said cannula.

9. The apparatus of claim 1, wherein said inlet is located near the distal end of said cannula, and wherein said outlet is located near the proximal end of said cannula.

10. The apparatus of claim 1, wherein said inlet includes a unidirectional valve that allows flow into said cannula and prevents flow out of said cannula.

11. The apparatus of claim 1, wherein said outlet includes a unidirectional valve that allows flow out said cannula and prevents flow into said cannula.

12. The apparatus of claim 1, further comprising a blood oxygenation sub-system associated with said pumping mechanism, able to oxygenate a patient's blood.

13. The apparatus of claim 1, further comprising a organ survival sub-system associated with said pumping mechanism, able to extend the life of an organ.

14. The apparatus of claim 1, further comprising a drug delivery sub-system associated with said pumping mechanism, able to deliver drugs to said cannula.

15. The apparatus of claim 1, further comprising a filtration sub-system associated with said pumping mechanism, able to remove excess fluid from blood.

16. The apparatus of claim 15, wherein said filtration sub-system comprises a semi-permeable diaphragm separating a first and a second compartment of said reservoir.

17. The apparatus of claim 1, wherein said filtration subsystem comprises at least one ultrafiltration agent.

18. The apparatus of claim 1, wherein said filtration sub-system is to enable reverse osmosis.

19. The apparatus of claim 1, wherein said filtration sub-system is to enable reverse ultrafiltration.

20. The apparatus of claim 2, wherein said reservoir further comprises a diaphragm of substantially non-permeable material between said proximal compartment and said distal compartment.

21. The apparatus of claim 2, wherein said reservoir further comprises a diaphragm of substantially semi-permeable material, to enable a flow of selected materials through said diaphragm.

22. The apparatus of claim 1, wherein said cannula has predetermined markings at one or more selected locations.

23. The apparatus of claim 1, wherein at least a portion of said cannula is coated with a chemical compound.

24. The apparatus of claim 1, wherein at least a portion of said reservoir is coated with a chemical compound.

25. A cannula suitable for insertion onto a blood system comprising an inlet and an outlet, said inlet and said outlet being at a predetermined distance from each other such that said inlet is located in proximity to a first organ, and said outlet is located in proximity to a second organ, when the cannula is inserted into said blood system.

26. The cannula of claim 25 having a proximal end connected to an extra corporeal blood pump.

27. The cannula of claim 25 wherein said valves are unidirectional.

28. The cannula of claim 25 comprising a blood filtration sub-system associated with said pumping mechanism, able to extract excess fluids from blood collected in the cannula.

29. The cannula of claim 25 comprising an oxygenation sub-system associated with the cannula, able to oxygenate blood collected in the cannula.

30. An apparatus comprising:
a cannula suitable for insertion onto a blood system having at least one inlet and at least one outlet;
a pumping mechanism able to pump blood through said cannula from said inlet to said outlet; and
a filtration sub-system able to filter blood delivered via said cannula.

31. The apparatus of claim 30, wherein said pumping mechanism includes a pulsafile pump.

32. The apparatus of claim 30, wherein said pumping mechanism includes an extra corporeal blood pump and a fluid reservoir, wherein said fluid reservoir comprises a proximal compartment associated with said pump and a distal compartment associated with a proximal end of said cannula.

33. The apparatus of claim 30, wherein when said cannula is inserted into a human body, said inlet being situated in the vicinity of a first organ, and said outlet being situated in the vicinity of a second organ, to receive blood from said first organ.

34. The apparatus of claim 30, comprising an oxygenation sub-system to oxygenate blood in said cannula.

35. The apparatus of claim 30, wherein said filtration subsystem it to enable ultrafiltration of blood delivered via said cannula.

36. The apparatus of claim 30, wherein said filtration sub-system it to enable reverse osmosis of blood delivered via said cannula.

37. An organ assist method, comprising:
insetting percutaneously a cannula into a peripheral artery, said cannula having an inlet situated substantially in proximity to a first organ, and an outlet situated substantially in proximity to a second organ;
receiving blood to said inlet from said first organ;
delivering blood to said second organ from said outlet.

38. The method of claim 37, wherein receiving of blood from said first organ comprises receiving blood from the vicinity of the aorta.

39. The method of claim 38 wherein said second organ comprises the kidneys.

40. The method of claim 37, comprising controlling said receiving and said delivering of said blood using a pumping mechanism.

41. The method of claim 37, comprising implementing filtration of blood delivered by said cannula.
